(19) United States
(12) Patent Application Publication
(10) Pub. No.: US 2026/0076637 A1
(43) Pub. Date: Mar. 19, 2026

(54) IMAGE CORRECTION METHOD AND MEDICAL X-RAY DEVICE

(71) Applicant: Siemens Healthineers AG, Forchheim (DE)

(72) Inventors: Philipp` Roser, Allston, MA (US); Alois Regensburger, Poxdorf (DE)

(21) Appl. No.: 19/328,086

(22) Filed: Sep. 12, 2025

(30) Foreign Application Priority Data
Sep. 13, 2024 (DE) 10 2024 208 713.0

(51) Int. Cl. A61B 6/00; A61B 6/42; G06T 5/60; G06T 5/70; G06T 5/73

(52) U.S. Cl. CPC A61B 6/5258; A61B 6/4233; A61B 6/482; G06T 5/60; G06T 5/70; G06T 5/73; G06T 2207/10116; G06T 2207/20021; G06T 2207/20192

(57) ABSTRACT

A method for correcting artifacts includes recording an X-ray image of an object by an X-ray detector. A primary X-ray spectrum of X-ray radiation is provided, and an off focal radiation spectrum in relation to the primary X-ray spectrum is provided. An image X-ray spectrum is determined from the X-ray image for each image pixel. One weighting factor each is determined by calculating a quotient between high-energy irradiation and overall energy irradiation for each image pixel, and a weighted image is determined from the weighting factors of the image pixels. A high-energy image is provided from high-energy components of the image X-ray spectrum, and low-energy components of the image X-ray spectrum are processed by taking into account image features and/or structures of the high-energy image. The high-energy component and the processed low-energy component are recombined for each image pixel, and a corrected X-ray image is created.

This application claims the benefit of German Patent Application No. DE 10 2024 208 713.0, filed on Sep. 13, 2024, which is hereby incorporated by reference in its entirety.

### BACKGROUND

The present embodiments relate to correcting artifacts.

What is known as off focal radiation (OFR) (e.g., X-ray radiation outside of the central ray) is a known problem with X-ray tubes in which electromagnetic fields are used to accelerate electrons to an anode made from a material with a high atomic number (e.g., tungsten). A large portion of the electrons is backscattered and accelerated again, although the portion then strikes the anode outside of the focal spot/focal path. Some of the electrons interact with the anode and generate unfocussed X-rays (e.g., about 10% of the emitted X-rays). In the case of imaging, these generate a slight fuzziness that lowers the spatial and intensity resolution. This affects, for example, tomographic reconstructions and impairs accuracy and sharpness of the Hounsfield units. For tungsten, which is used in angiography and computed tomography, up to 50% of the electrons are backscattered. For materials with lower atomic numbers, as are used, for example, in mammography, less than 40% are backscattered. In FIG. 3, the scatter coefficient n in percent is plotted as a graph against the electron energy E₀ for a plurality of materials (e.g., tungsten (W) and copper (Cu)).

To reduce the off focal radiation, improvements in the electromagnetic fields, by way of example, are sought in the prior art in order to improve the efficiency of the X-ray tubes. Alternatively, an approach is known in which attempts are made to remove the off focal radiation from the recorded images with the aid of simulations.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method that makes improved imaging with regard to off focal radiation possible is provided. As another example, an X-ray device suitable for carrying out the method is provided.

The method of the present embodiments for correcting artifacts, generated by off focal radiation (=OFR), in an X-ray image of an object includes the following acts: recording the X-ray image of the object by an X-ray detector with a large number of detector pixels, which object is radiographed by X-ray radiation emitted by an X-ray tube; providing a primary X-ray spectrum of the X-ray radiation and providing an estimated or ascertained off focal radiation spectrum in relation to the primary X-ray spectrum; determining an image X-ray spectrum from the X-ray image for each image pixel; dividing the image X-ray spectrum into a high-energy component and a low-energy component for each image pixel; determining one weighting factor each by calculating the quotient between high-energy irradiation and overall energy irradiation for each image pixel and determining a weighted image from the weighting factors of the image pixels; providing a high-energy image from the high-energy components; processing the low-energy components for each image pixel with regard to the off focal radiation portion by at least one image-processing algorithm by taking into account image features and/or structures of the high-energy image; and recombining the high-energy component and the processed low-energy component for each image pixel and creating a corrected X-ray image therefrom.

The method creates a possibility for correcting impairments, generated by off focal radiation, of the image quality of X-ray images and for obtaining a particularly precise and good image quality. A high image quality brings about an improved diagnosis as well as improved care of patients. The method utilizes the fact that the off focal radiation particularly contains low-energy photons since the generating electrons have a low energy. For this reason, each X-ray image includes a high-energy component that is substantially free from off focal radiation. Skillful division of the energy components of the X-ray image makes it possible to then process the severely impaired low-energy-component accordingly such that with regard to the items of image information, it is adjusted to the high-energy component that is not impaired.

I represents the respective radiation intensity. 0<w<1 applies to the individual weighting factors w. The physical variable radiation Hₑ is generally indicated in the units J·m⁻² (e.g., radiant fluence or radiant exposure).

According to one embodiment, the primary X-ray spectrum is calculated from the X-ray voltage, the X-ray current, the filtering of the X-ray radiation, and the position of the image pixels, where u, v represent the positions of the image pixels in the pixel matrix of the X-ray detector. A calculation of this kind is known and may be easily carried out. A particularly precise calculation brings about particularly good image quality.

According to a further embodiment, the off focal radiation spectrum is determined by simulation (e.g., Monte Carlo simulation) or by a machine learning algorithm. Monte Carlo simulations are capable of calculating even complicated scenarios very precisely. The calculations may be determined quickly and exactly by machine learning. A precise determination of the spectrum of the interfering off focal radiation constitutes a prerequisite for optimum correction of the impairments of the X-ray image.

According to a further embodiment, the determination of an image X-ray spectrum for each image pixel is carried out using a division of the X-ray image into at least one bone density image D_{b}(u, v) and one water density image D_{w}(u, v) and using the primary X-ray spectrum and the off focal radiation spectrum. This constitutes a particularly exact and advantageous method of division in medical imaging since a human or animal body may be approximated via these two main elements. Methods of the present embodiments for the division of the X-ray image into at least one bone density image and one water density image are formed, by way of example, by bone structure constructions based on detected image contents or by a method using patient convexity conditions. In the latter case, it is assumed that the shape of the patient is convex, smooth, and continuous (CSC), and this is used for the estimation that the object (e.g., the patient or a part of them) is constructed of basis materials, such as bones and water. Methods for such a division are basically prior art.

The determination of the image X-ray spectrum may be carried out using the known polyenergetic Beer-Lambert law. The Beer-Lambert law describes the attenuation of the intensity of radiation with respect to the initial intensity thereof as it passes through an object as a function of the material properties of the object. This represents a known and proven method.

According to a further embodiment, the first threshold value between high energy and low-energy is selected such that the off focal radiation portion of the high-energy component is negligible. This may be configured, for example, such that the off focal radiation portion of the high-energy component falls below a limit value (e.g., a specific absolute value or a percentage, such as 2% or 5%). The limit value may be selected beforehand by an operator or be automatically set. It is advantageous to select an optimally low limit value, but one at which a significant high-energy component with sufficiently relevant items of image information still exists. The values refer, for example, to the calculated or simulated spectra.

In one embodiment, the high-energy image (e.g., the image composed of the high-energy components for the individual image pixels in accordance with the pixel matrix) may be represented based on the weighted image W(u, v) and the X-ray image I(u,v) according to the following formula: Iₕ(u, v)=I(u, v)·W(u, v). The weighted image is constructed from a matrix, in accordance with the pixel matrix of the X-ray detector, from weighting factors between 0 and 1. The high-energy image may likewise show a mapping of structures of the object without blurring due to interfering off focal radiation.

The processing or correction of the low-energy components by taking into account image features and/or structures that may be detected in the high-energy image/the high-energy components, such as corners or edges, results in a significant improvement in the image quality. The image features and structures in the high-energy image may be identified, for example, by segmentation. Since scarcely any or even no artifacts or fuzziness due to off focal radiation exists in the high-energy image, a correction and/or modeling of the low-energy component according to the model of the high-energy component is a particularly effective method for reducing or eliminating the effect of the off focal radiation and thereby for optimizing the image quality.

According to a further embodiment, the at least one image-processing algorithm for processing or correcting the low-energy components is an edge reinforcement algorithm and/or a guided filter algorithm (e.g., smoothing filter that preserves edges). Algorithms of this kind are known and proven in the processing of X-ray images and may be used quickly and easily. Any other algorithms that may affect an image improvement may also be used.

According to a further embodiment, at least one substep or act of the method is executed by machine learning (e.g., the creation of the off focal radiation spectrum, the image processing/correction of the low-energy component, or the division of the X-ray image).

The present embodiments also include a medical X-ray device for carrying out a method as described above, having an X-ray detector with a large number of image pixels for recording an X-ray image of an object radiographed by X-ray radiation, an X-ray tube with a cathode and an anode for generating X-ray radiation that also generates off focal radiation, a control unit for actuating the acts of the method, a calculation unit, and an image processing unit with at least one algorithm for processing at least one X-ray image (e.g., at least one algorithm for machine learning).

### BRIEF DESCRIPTION OF THE DRAWINGS

FIG. 1 shows a sequence of acts of an embodiment of a method for correction of artifacts generated by off focal radiation;

FIG. 2 shows a view of a typical energy spectrum for a tungsten anode;

FIG. 3 shows a view of a scatter coefficient n as a function of electron energy for different materials; and

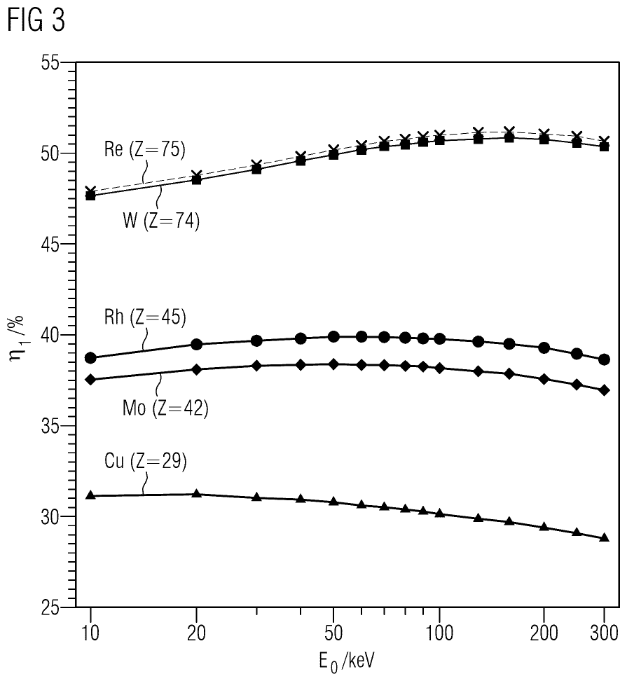

FIG. 4 shows a view of an embodiment of an apparatus for carrying out the method.

### DETAILED DESCRIPTION

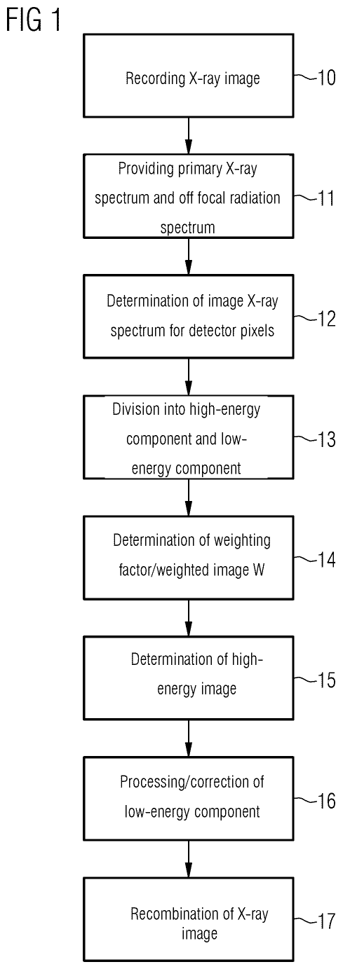

FIG. 1 shows acts of an embodiment of a method for correction of artifacts, generated by off focal radiation (=OFR), in an X-ray image of an object. Since electrons that result in the generation of the off focal radiation cause X-ray photons with, on average, lower energy, a high-energy component of the X-ray image exists that is substantially free from off focal radiation. The basic idea of the method consists in utilizing the items of information of the (e.g., for the most part unaffected) high-energy component in order to correct the effects of the off focal radiation that adversely affects the low-energy component.

An X-ray image I(u, v) is recorded in a first act 10. For this, an object (e.g., a patient or part/organ of a patient) is X-rayed by X-ray radiation generated by an X-ray tube of an X-ray device. The X-ray radiation is characteristically attenuated as a result and detected by an X-ray detector and converted into an X-ray image. The X-ray detector is constructed from a matrix with rows and columns of detector pixels (u, v), where u and v represent the respective positions of the detector pixels. Each detector pixel generates an image pixel in the X-ray image.

A primary X-ray spectrum I₀(u, v, E) of the X-ray radiation of the X-ray tube and an off focal radiation spectrum in relation to the primary X-ray spectrum are provided in a second act 11. The second act may also be carried out before the first act already if all X-ray parameters, with which the X-ray image is recorded, are known. The primary X-ray spectrum I₀(u, v, E) may be determined from the tube voltage of the X-ray tube, the tube current, the filtering of the X-ray radiation, and the position of the image pixels, where u, v represent the position of the image pixels and E the energy. The off focal radiation spectrum may be estimated or calculated from the primary X-ray spectrum (e.g., by simulation (Monte Carlo simulation)), or be ascertained by a machine learning algorithm. Both spectra may either be determined/calculated directly or be ascertained in advance, be extracted from a memory, and be provided.

An image X-ray spectrum I_{S}(u, v, E) is subsequently determined in a third act 12 for those image pixels of the X-ray detector that were used for imaging the X-ray image. This may be carried out in different ways. A particularly precise method is based on use of the Beer-Lambert law using the primary X-ray spectrum, the off focal spectrum, and a breakdown of the X-ray image into basis materials (e.g., into at least one bone density image D_{b}(u, v) and one water density image D_{w}(u, v)). For example, this is carried out for each image pixel of the X-ray image.

Methods for breaking down into a bone density image and a water density image are basically known. For example, a method that draws on the use of patient convexity conditions may be used, with it being assumed, for example, that the shape of the patient is convex, smooth, and continuous (CSC); this is then used for the assessment that the object (e.g., the patient or a part of them) is constructed from basis materials such as bone and water. See further below under CSC method for a more detailed description of these methods.

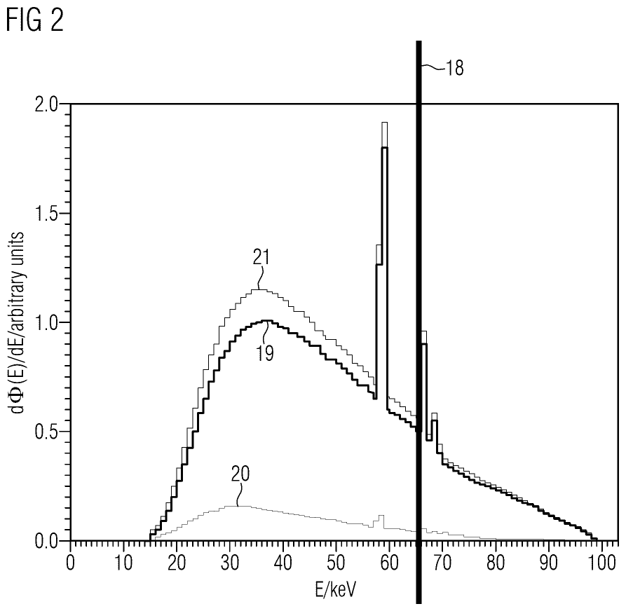

The image X-ray spectrum for each image pixel is divided into a high-energy component and a low-energy component in a fourth act 13. The first threshold value that separates the high energy from the low-energy is selected such that the off focal radiation portion of the high-energy component is negligible, for example, with respect to the off focal radiation portion in the region of the low-energy component. The first threshold value may be defined in advance, be selected by an operator, or be automatically set. Thus, for example, the first threshold value 18 may be used in place of the energy (see, e.g., x-axis in FIG. 2), at which the percentage of the off focal radiation spectrum 20 drops below a limit value when compared with the overall spectrum 21 (e.g., primary energy spectrum 19 plus off focal radiation spectrum 20; 5% or 2% or 1%) or if the absolute value of the off focal radiation or the standardized off focal radiation drops below a limit value. FIG. 2 shows one example of the first threshold value 18 of the energy. In one embodiment, an optimally low first threshold value is selected, but one at which a significant high-energy component with sufficiently relevant items of image information still exists. In the example shown in FIG. 2 for a tungsten anode of an X-ray tube, the first threshold value 18 is approximately 65 keV: the low-energy component goes up to 64 keV, for example, and above 65 keV the high-energy component. Φ(E) is the radiant power (e.g., radiant energy per unit of time). The braking radiation peaks may, for example, as absolute values, be left out. FIG. 2 shows a typical overall spectrum 21, total of a typical primary radiation spectrum 19, and a typical off focal radiation spectrum 20.

One weighting factor wᵤᵥ each is determined for the corresponding image pixels in a fifth act 14 by calculation of the quotient between the high-energy irradiation and the overall energy irradiation (e.g., by a calculation unit). The physical variable radiation Hₑ is generally indicated in the units J·m⁻² (e.g., radiant energy per surface, radiant fluence, or radiant exposure). The weighting factors wᵤᵥ lie between 0 and 1 (e.g., 0<wᵤᵥ<1) and indicate the portion of radiant energy per surface of the quasi off focal radiation-free high-energy component in the overall radiant energy per surface. A weighted image W(u, v) may then be determined from the weighting factors wᵤᵥ for the individual image pixels.

A high-energy image is subsequently provided for each image pixel in a sixth act 15 using the high-energy component. This may also be represented as follows: Iₕ(u, v)=I(u, v)·W(u, v). The high-energy image shows a mapping of features and structures of the object without blurring and without artifacts that are generated by the off focal radiation and therefore has a high image quality.

The low-energy component Iₗ(u, v) of the image X-ray spectrum is corrected in a seventh act 16 with regard to the off focal radiation portion by taking into account the high-energy image created in the sixth act 15. Image processing algorithms and/or correction algorithms, for example, are used for this. Machine-learning algorithms may also be used. The image processing algorithms may be, for example, an edge reinforcement algorithm and/or a guided filter algorithm (e.g., smoothing filter that preserves edges) or another known optimization algorithm. The low-energy component is processed, for example, such that the resulting structures correspond to the structures in the high-energy image (e.g., with regard to edges, fuzziness, and other known adverse effects of the off focal radiation on the image quality). The low-energy component may be modeled during processing with respect to the off focal radiation portion (e.g., in accordance with the high-energy component). Items of information that are gathered from the high-energy image, such as located structures or features, are used for the correction of the respective low-energy components. The adverse effects and artifacts of the off focal radiation may also be particularly effectively reduced in the low-energy components via the existing high-energy image as a model/template.

The processed, corrected low-energy component and the (unprocessed) high-energy component are subsequently merged again in an eighth act 17, and an improved, corrected X-ray image is thereby obtained in which the adverse effects of the off focal radiation are significantly reduced. The image quality is thereby considerably higher, and good diagnoses and/or therapies may be provided, even with a strong off focal radiation of an X-ray source, based on the X-ray images. The outlay of the present method is low, and the improvement in the image quality considerable.

In addition, the method may be iteratively carried out in that the third to eight acts are repeated a number of (e.g., several) times. This is helpful, for example, since the off focal radiation of adjacent detector pixels may have an effect on the image quality. Algorithms based on an expectation-maximization or other optimization algorithms may also be used for this.

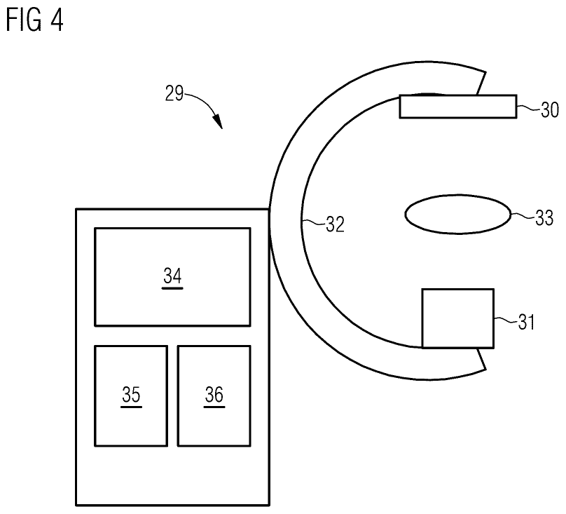

FIG. 4 shows a medical X-ray device 29 that is configured for carrying out the method. The medical X-ray device 29 has an X-ray detector 30 with a large number of image pixels for recording an X-ray image of an object 33 radiographed by X-ray radiation, and an X-ray tube 31 with a cathode and an anode for generating X-ray radiation that also generates off focal radiation. The X-ray detector 30 and the X-ray tube 31 may be mounted, for example, by an adjustable C-arm 32. The medical X-ray device also has a control unit 34 for actuating the acts of the method, a calculation unit 35, and an image processing unit 36 with at least one algorithm for processing at least one X-ray image. A typical medical X-ray device 29 of this kind may be, for example, a cone beam CT (CBCT), a fluoroscopy X-ray device, an angiography X-ray device, and/or a CT.

As an alternative or in addition to the pixel-by-pixel processing, adjacent image pixels may also be incorporated. Thus, for example, adjacent image pixels (e.g., or the irradiation striking them) may also be incorporated, for example, in the calculation of the quotient between the high-energy irradiation and the overall energy irradiation instead of a pixel-by-pixel calculation. Adjacent image pixels may also be taken into account in the correction of the low-energy components.

The first threshold value may vary spatially in order to be adjusted to different regions in which a clearly different off focal radiation is expected. A plurality of threshold values may accordingly also be used for different regions.

When an X-ray detector with spectral resolution is used, as an alternative to the calculation of the pixel-by-pixel X-ray spectra, the X-ray spectra may also simply be gathered from the X-ray detector (e.g., in the case of a multi-layer X-ray detector or a counting (photon-counting) X-ray detector). Corresponding X-ray devices are, for example, computer tomographs with counting or multi-layer fan beam detectors.

Method that uses patient convexity conditions (CSC method). First, the following conditions apply with this approach: since, in general, examination objects are positioned in the X-ray beam with the smallest possible path length (e.g., no avoidable other objects in the way), it may be assumed that the shape of the patient is convex, smooth, and continuous (CSC). It is also assumed that the imaging properties of the examination object may be sufficiently well described by basis materials (mat) such as bone and water. The line integral P of the linear attenuation may then be determined from the recorded X-ray images using an intensity normalization and a log transformation. The line integrals per pixel may be regarded as the total of the expected material attenuation coefficient μ̅ₘₐₜ and the path length lₘₐₜ. The CSC conditions are used since the mapping of the line integrals to the path lengths cannot be unambiguously solved. The implementation of this approach may be carried out as follows:
1. Recording an X-ray projection image I_{raw}∈ [symbol] ²
2. Carrying out an intensity normalization:

$$I = \frac{{I}_{raw}}{{I}_{0}}$$

3. Carrying out a log transformation, for example P=−log (I), this produces the line integrals of the overall attenuation for all pixels
4. Determination of expected linear attenuation coefficients as a function of the parameters of the X-ray tube, for example μ̅ₘ̅ₐ̅ₜ̅= [symbol] [μₘₐₜ(E)], where E is the photon energy
5. Breaking down the line integrals into known, expected linear attenuation coefficients and associated path lengths: P=Σₘₐₜμ̅ₘₐₜ·lₘₐₜ, where the path length lₘₐₜ∈ [symbol] ²; with the limitation that the total path length or the object thickness l=Σₘₐₜlₘₐₜ satisfies the CSC conditions, results as P=μ̅_{bone}·l_{bone}+μ̅_{water}·l_{water}. Breaking down may be carried out by known optimizers (e.g., gradient descent). At the beginning, the path length may be set, for example, to zero for all materials except one.

Independent of the grammatical term usage, individuals with male, female, or other gender identities are included within the term.

The present embodiments may be briefly summarized as follows: for particularly good image quality, a method for correcting artifacts, generated by off focal radiation (=OFR), in an X-ray image of an object with the following acts is provided: recording the X-ray image of the object by an X-ray detector with a large number of detector pixels, which object is radiographed by X-ray radiation emitted by an X-ray tube; providing a primary X-ray spectrum of the X-ray radiation and providing an estimated or ascertained off focal radiation spectrum in relation to the primary X-ray spectrum; determining an image X-ray spectrum from the X-ray image for each image pixel; dividing the image X-ray spectrum into a high-energy component and a low-energy component for each image pixel; determining one weighting factor each by calculating the quotient between high-energy irradiation and overall energy irradiation for each image pixel and determining a weighted image from the weighting factors of the image pixels; providing a high-energy image from the high-energy components; processing the low-energy components for each image pixel with regard to the off focal radiation portion by at least one image-processing algorithm by taking into account image features and/or structures of the high-energy image; and recombining the high-energy component and the processed low-energy component for each image pixel and creation of a corrected X-ray image therefrom.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for correcting artifacts, generated by off focal radiation, in an X-ray image of an object, the method comprising:
recording the X-ray image I(u, v) of the object by an X-ray detector with a number of detector pixels, the object being radiographed by X-ray radiation emitted by an X-ray tube;
providing a primary X-ray spectrum I₀(u, v, E) of the X-ray radiation and providing an estimated or ascertained off focal radiation spectrum in relation to the primary X-ray spectrum;
determining an image X-ray spectrum I_{S}(u, v, E) from the X-ray image for each image pixel;
dividing the image X-ray spectrum I_{S}(u, v, E) into a high-energy component Iₕ(u, v) and a low-energy component Iₗ(u, v) for each image pixel;
determining a weighting factor wᵤᵥ, the determining of the weighting factor wᵤᵥ comprising calculating a quotient between high-energy irradiation and overall energy irradiation for each image pixel and determining a weighted image W(u, v) from the weighting factors wᵤᵥ of the image pixels;
providing a high-energy image from the high-energy components Iₕ(u, v);
processing the low-energy components Iₗ(u, v) for each image pixel with regard to the off focal radiation portion using at least one image-processing algorithm, the processing comprising taking into account image features, structures, or the image features and the structures of the high-energy image; and
recombining the high-energy component and the processed low-energy component for each image pixel and creating a corrected X-ray image therefrom.

2. The method of claim 1, wherein dividing the image X-ray spectrum for each image pixel into a high-energy component and a low-energy component comprises selecting a first threshold value between high energy and low energy, such that the off focal radiation portion of the high-energy component is negligible.

3. The method of claim 1, wherein the primary X-ray spectrum is calculated from an X-ray voltage, an X-ray current, a filtering of the X-ray radiation, and a position of the image pixels, and
wherein u, v represent positions of the image pixels.

4. The method of claim 1, wherein the off focal radiation spectrum is determined by simulation or by a machine learning algorithm.

5. The method of claim 4, wherein the simulation is a Monte Carlo simulation.

6. The method of claim 1, wherein determining the image X-ray spectrum for each image pixel is carried out using a division of the X-ray image into at least one bone density image and one water density image and using the primary X-ray spectrum and the off focal radiation spectrum.

7. The method of claim 6, wherein dividing the X-ray image into the at least one bone density image and the water density image is carried out using patient convexity conditions or using bone structure constructions based on detected image contents.

8. The method of claim 6, wherein determining the image X-ray spectrum is carried out using the Beer-Lambert law.

9. The method of claim 2, wherein the first threshold value between high energy and low energy is selected such that the off focal radiation of the high-energy component drops below a limit value.

10. The method of claim 1, wherein the high-energy components Iₕ(u, v) are representable as: Iₕ(u, v)=I(u, v)·W(u, v).

11. The method of claim 1, wherein the at least one image-processing algorithm comprises an edge reinforcement algorithm, a guided filter algorithm, or the edge reinforcement algorithm and the guided filter algorithm.

12. The method of claim 1, wherein the recording, the providing of the primary X-ray spectrum of the X-ray spectrum of the X-ray radiation and the providing of the estimated or ascertained off focal radiation spectrum, the determining of the image X-ray spectrum, the dividing, the determining of the weighting factor, the providing of the high-energy image, the processing, the recombining, or any combination thereof is executed using machine learning.

13. A medical X-ray device comprising:
an X-ray detector with a number of image pixels for recording an X-ray image of an object radiographed by X-ray radiation;
an X-ray tube comprising a cathode and an anode, the X-ray tube being configured to generate X-ray radiation that also generates off focal radiation,
a controller configured to:
record the X-ray image I(u, v) of the object by the X-ray detector with the number of detector pixels, the object being radiographed by the X-ray radiation emitted by the X-ray tube;
provide a primary X-ray spectrum I₀(u, v, E) of the X-ray radiation and provide an estimated or ascertained off focal radiation spectrum in relation to the primary X-ray spectrum;
determine an image X-ray spectrum I_{S}(u, v, E) from the X-ray image for each image pixel;
divide the image X-ray spectrum I_{S}(u, v, E) into a high-energy component Iₕ(u, v) and a low-energy component Iₗ(u, v) for each image pixel;
determine a weighting factor wᵤᵥ, the determination of the weighting factor wᵤᵥ comprising calculation of a quotient between high-energy irradiation and overall energy irradiation for each image pixel and determination of a weighted image W(u, v) from the weighting factors wᵤᵥ of the image pixels;
provide a high-energy image from the high-energy components Iₕ(u, v);
process of the low-energy components Iₗ(u, v) for each image pixel with regard to the off focal radiation portion using at least one image-processing algorithm, the process comprising taking into account image features, structures, or the image features and the structures of the high-energy image; and
recombine the high-energy component and the processed low-energy component for each image pixel and create a corrected X-ray image therefrom;
a calculation unit; and
an image processing unit with at least one algorithm for processing at least one X-ray image.

14. The medical X-ray device of claim 13, further comprising at least one algorithm for machine learning.
